(19) United States
(12) Patent Application Publication
(10) Pub. No.: US 2022/0400939 A1
(43) Pub. Date: Dec. 22, 2022

(54) VARIABLE/SELF-SECURING EAR SPECULUM

(71) Applicant: Kasra Rastani, San Francisco, CA (US)

(72) Inventor: Kasra Rastani, San Francisco, CA (US)

(21) Appl. No.: 17/845,775

(22) Filed: Jun. 21, 2022

Related U.S. Application Data
(60) Provisional application No. 63/202,644, filed on Jun. 18, 2021.

(51) Int. Cl. A61B 1/227; A61B 1/32

(52) U.S. Cl. CPC A61B 1/227; A61B 1/32

(57) ABSTRACT

A speculum includes, in part, an iris aperture. The iris aperture includes, in part, an actuator and a multitude of blades. Each blade is adapted to vary the opening of the aperture in response to an applied force. Each blade includes, in part, a wing extending outwardly from the aperture. Each wing has a base that is larger than the wing's tip. Each wing has a curved surface along at least a portion of the wing's height. In one embodiment, the at least portion of the wing's height is the entirety of the wing's height. Each blade and its associated wing are optionally formed from the same material. Therefore, a single piece of the material may be shaped to include both the blade and its associated wing. The blades may optionally be made from a material that is different from the material from which the wings are made.

### RELATED APPLICATION

The present application claims benefit under 35 USC 119(e) of U.S. patent application Ser. No. 63/202,644, filed Jun. 21, 2021, the content of which is incorporated herein by reference in its entirety.

### TECHNICAL FIELD

The present application relates to an ear speculum, and more particularly to a self-securing ear speculum.

### BACKGROUND

Ear specula are well known and widely used to examine a patient's ear. They are also used to perform ear surgery by otolaryngologists. The diameter of the ear canal of a patient depends on his/her age, anatomy and many other factors. The opening of a conventional ear speculum has a fixed diameter, often ranging from 2-6 mm. FIG. 1 shows specula 10, 12, 14, 16, 18 and 20 each of has a different opening size and/or a different length L. Depending on the ear canal size of the patient, a speculum with the appropriate size is selected for use.

During an examination, one hand of a doctor/user usually holds the speculum in place, while the other hand performs an operation, such as debridement. To free up the hand holding the speculum for bimanual manipulation during an ear surgery, an articulating speculum holder, such as articulating speculum 20 shown in FIG. 2, may be used to keep the ear speculum in place. The articulating arm, which is typically around 30 cm long, is secured to the side of the operating table with a stand and a clamp, thus rendering the process cumbersome. When the speculum needs to be adjusted, or to accommodate changes in the visualization angle, the entire set up has to be loosened, adjusted, and then retightened, thus complicating and prolonging the surgery.

### SUMMARY

A speculum, in accordance with one embodiment of the present disclosure, includes, in part, an iris aperture that includes, in part, an actuator and a multitude of blades. Each blade is adapted to vary the opening of the aperture in response to a force applied to the actuator. Each blade includes, in part, a wing extending outwardly from the aperture. Each wing has a base that is larger than the wing's tip. Each wing has a curved surface along at least a portion of the wing's height. In one embodiment, the at least portion of the wing's height is the entirety of the wing's height. In one embodiment, the speculum includes, in part, 4 blades and 4 wings. In another embodiment, the speculum includes, in part, 5 blades and 5 wings.

In one embodiment, each blade and its associated wing are formed from the same material. Therefore, in such embodiment, a single piece of the material is shaped to include both the blade and its associated wing. In one embodiment, the blades are made from a first material that is different from a second material from which the wings are made. In one embodiment, the material from which the blades and the wings are made is selected from a group consisting of plastic and stainless steel.

In one embodiment, after being inserted into a patient's ear canal, a force is applied to the actuator thereby causing the wings to rotate and increase the opening between the wing's tips. The force further causes the wings to make contacts with the tissue in the patient's ear canal so as to securely hold the speculum in place. In one embodiment, the iris aperture is a friction-based aperture adapted to adjust and maintain the aperture's opening using friction so as to lock the plurality of wings when inserted into a patient's ear. In one embodiment, the iris aperture operates using a ratchet mechanism. The ratchet mechanisms is used to adjust and maintain the aperture's opening so as to lock the plurality of wings when inserted into a patient's ear.

A method of forming a speculum, in accordance with one embodiment of the present disclosure includes, in part, forming an iris aperture that includes, in part, an actuator and a multitude of blades. Each blade is adapted to vary the opening of the aperture in response to a force applied to the actuator. Each blade includes, in part, a wing extending outwardly from the aperture. Each wing has a base that is larger than the wing's tip. Each wing has a curved surface along at least a portion of the wing's height. In one embodiment, the at least portion of the wing's height is the entirety of the wing's height. In one embodiment, the speculum includes, in part, 4 blades and 4 wings. In another embodiment, the speculum includes, in part, 5 blades and 5 wings.

In one embodiment, each blade and its associated wing are formed from the same material. Therefore, in such embodiment, a single piece of the material is shaped to include both the blade and its associated wing. In one embodiment, the blades are made from a first material that is different from a second material from which the wings are made. In one embodiment, the material from which the blades and the wings are made is selected from a group consisting of plastic and stainless steel.

In one embodiment, after being inserted into a patient's ear canal, a force is applied to the actuator thereby causing the wings to rotate and increase the opening between the wing's tips. The force further causes the wings to make contacts with the tissue in the patient's ear canal so as to securely hold the speculum in place.

### BRIEF DESCRIPTION OF THE DRAWINGS

FIG. 1 shows a number of specula as known in the prior art.

FIG. 2 shows an articulating speculum holder adapted to secure an ear speculum in place during an operation on a patient's ear, as known in the prior art.

FIG. 3A is a highly simplified view of an example of an iris aperture in a fully opened state.

FIG. 3B is a highly simplified view of an example of an iris aperture in a partially opened state.

FIG. 4 is a highly simplified perspective view of a speculum, in accordance with one exemplary embodiment of the present disclosure.

FIG. 5A is a schematic front view of a wing associated with a blade of an iris aperture of an ear speculum, in accordance with one exemplary embodiment of the present disclosure.

FIG. 5B is a side view of a wing associated with a blade of an iris aperture of an ear speculum, in accordance with one exemplary embodiment of the present disclosure.

FIG. 5C is a side view of a wing associated with a blade of an iris aperture of an ear speculum, in accordance with one exemplary embodiment of the present disclosure.

FIG. 6 is a perspective view of a speculum when the speculum's aperture is in a fully closed position, in accordance with one exemplary embodiment of the present disclosure.

FIG. 7 is a perspective view of the speculum of FIG. 6 when the speculum's aperture is in a partially open position, in accordance with one exemplary embodiment of the present disclosure.

FIGS. 8A and 8B are top and side views, respectively, of an speculum when the speculum's aperture is in a fully open position, in accordance with one exemplary embodiment of the present disclosure.

FIGS. 8C and 8d are top and side views, respectively, of the speculum shown in FIGS. 8A and 8B, when the speculum's aperture is in a closed position.

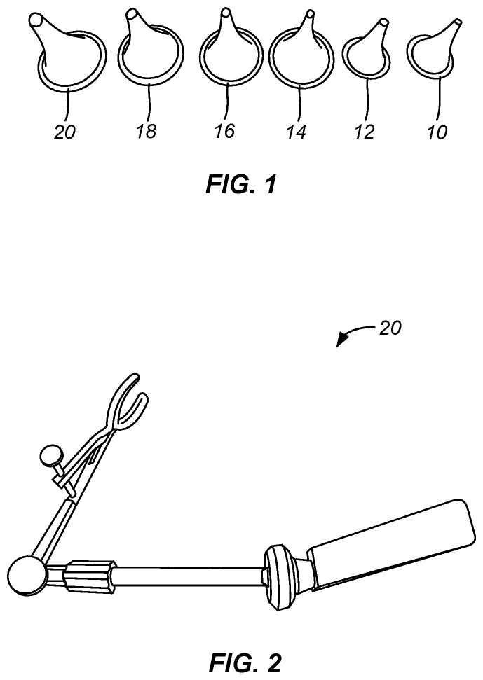

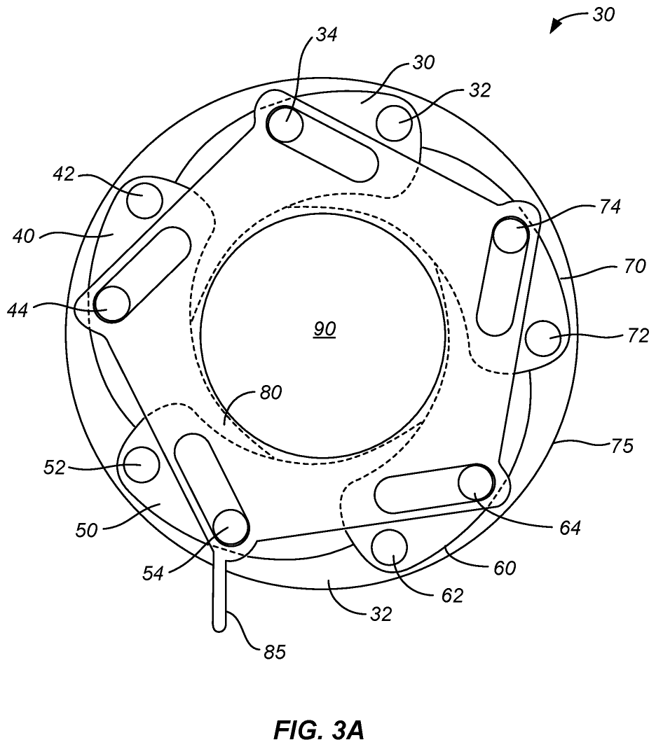

### DETAILED DESCRIPTION

A variable, self-adjusting, self-securing ear speculum, in accordance with one embodiment of the present disclosure, includes, in part, an iris aperture (also referred to herein as diaphragm) and a multitude of blades (also referred to herein as leaves) each of which includes a tapered (flared) outwardly expanding wing. The wings are tapered and may be curved such that when the aperture blades are brought in proximity of one another, the tapered wings form a substantially funnel-shape structure with openings at both ends of the structure.

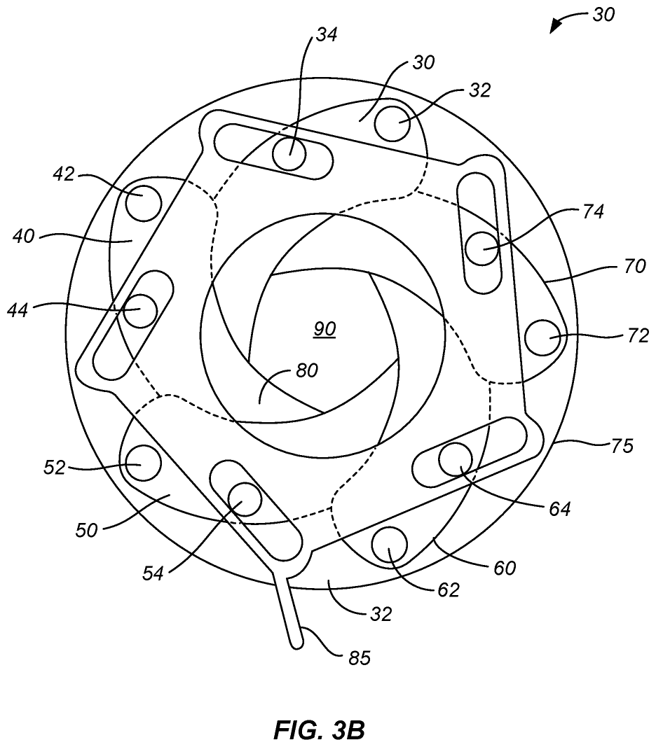

FIG. 3A is a highly simplified partial view of an example of an iris aperture 30 in a fully open state, in accordance with one embodiment of the present disclosure. Iris aperture 30 is shown as including, in part, a base plate 75, five exemplary blades 30, 40, 50, 60, 70, and a blade actuating ring 80. Each blade is secured to the base plate using an associated pin. In the example shown in FIG. 3A, blades 30, 40, 50, 60 and 70 are shown as being secured to base plate 75 using pins 32, 42, 52, 62 and 72. Associated with each blade is a second pin that causes the blade to rotate in response to the actuation of ring 80 via arm 85. Accordingly, by actuating arm 85, ring 80 causes blades 30, 40, 50, 60, 70 to rotate using pins 34, 44, 54, 64 and 74, respectively. The rotation of the blades, in turn, causes the opening 90 of the aperture to increase or decrease. FIG. 3B shows iris aperture 30 of FIG. 3A after the blades are caused to rotates so as to decrease opening 90 of the aperture. In other words, in FIG. 3B, iris aperture 30 is placed in a partially closed state.

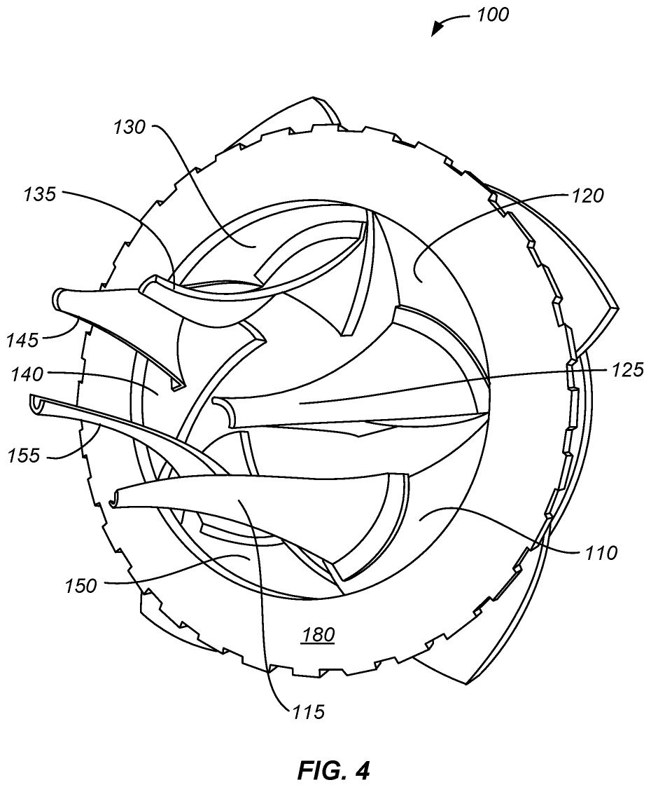

FIG. 4 is a highly simplified perspective view of a speculum 100, in accordance with one exemplary embodiment of the present disclosure. Speculum 100 includes, in part, an iris aperture as described above with reference to FIGS. 3A and 3B, formed on baseplate 180. Speculum 100 is shown as having five blades 110, 120, 130, 140 and 150. Although speculum 100 is shown as having five blades, it is understood that embodiments of the present disclosure are not so limited, and that an speculum, in accordance with embodiments of the present disclosure, may have any number of blades equal to or greater than 2.

Each blade has disposed thereon an associated wing that is curved, tapered and extends away from baseplate 180. The wings are tapered such that the width of each wing at the wing's base (i.e., where it is in contact with the wing's associated blade) is greater than the width of the wing at the wing's tip, i.e., where the wing makes a contact with a patient's inner ear when inserted therein. In one embodiment, the iris aperture is friction-based and thus uses friction to increase or decrease the aperture's opening. Accordingly, in such embodiment, friction is used to lock the blades, and thereby the wings in the patient's ear canal. In another embodiment, the iris aperture has a rachet mechanism to increase or decrease the aperture's opening; therefore, in such embodiments, the rachet mechanism causes the blades, and thereby the wings to remain locked in the patient's ear canal.

In one embodiment, each blade and its associated wing (e.g., blade 120 and its associated wing 125) are formed from a same material, such as stainless steel, or plastic. Therefore, in such embodiments, the same continuous piece of material is shaped to include a blade and its associated wing. In other embodiments, the wings and blades are made separately and coupled to one another during an assembly process. In one embodiment, the wings may be mated to the blades through a mating mechanism. In other embodiments, an adhesive may be uses to couple the wings to their respective blades. In one embodiment, the blades may be made from a first material that is different from a second material from which the wings are made.

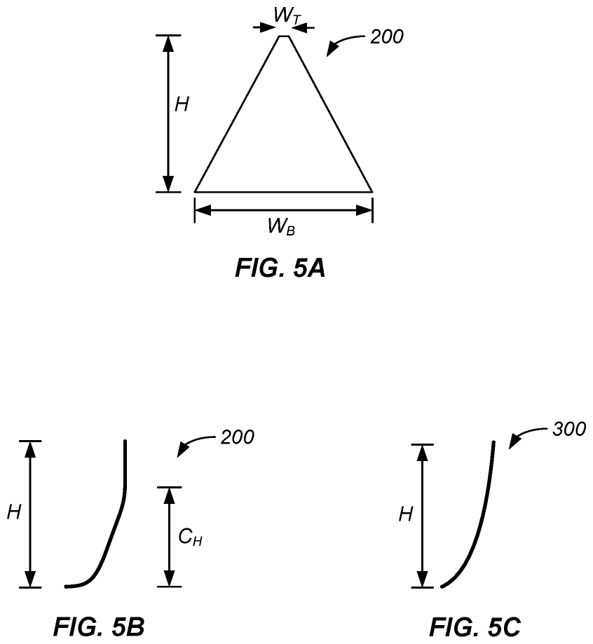

FIG. 5A is a front view of a wing 200 associated with a blade, in accordance with one exemplary embodiment of the present disclosure. Wing 200, which may correspond to the wings shown in FIG. 4, may have a wing base WB of 2-4 cm and a wing tip W_{T} of 1-3 mm.

To help ease the insertion and avoid discomfort to patients, the wings may be curved along a portion of their height H. FIG. 5B is a side view of wing 200, in accordance with one embodiment of the present disclosure. As is seen from FIG. 5B, wing 200 has a curvature along a portion of its height C_{H}. The curvature may be defined, for example, approximately by a parabola or an ellipse. Accordingly, when the aperture of the speculum is partly closed and the wings are brought into close proximity of one another, the wings form, for example, a partial paraboloid, ellipsoid or a funnel-shaped surface, along their height C_{H}. In other embodiments, the wings may be curved along substantially the entirety of their height H. FIG. 5C is a side view of a wing 300 which is curved along substantially an entirety of its height H. In one embodiment, each wing has a height of 4 cm.

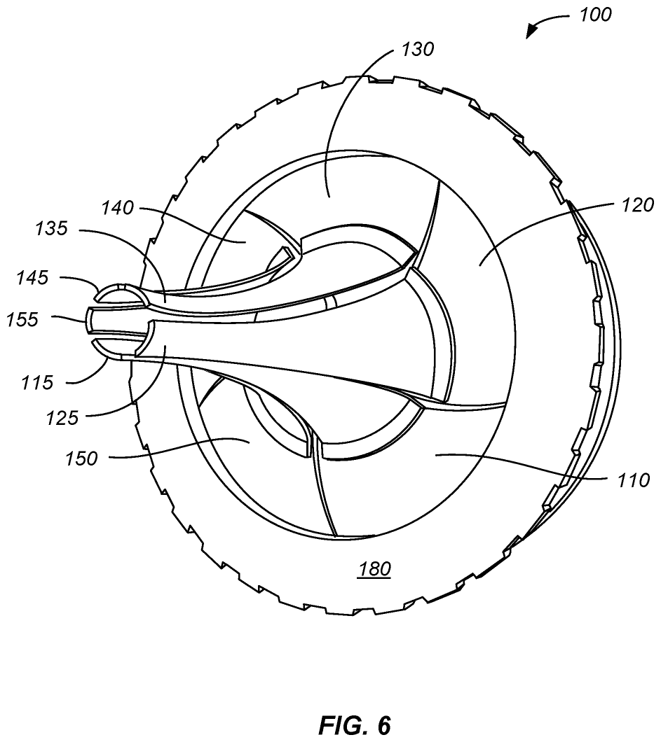

Prior to being inserted into a patient's ear canal, the speculum's aperture is partially or fully closed so as to bring the wings close to one another. By closing the aperture and thereby positioning the wings into close proximity of one another, the wings form a narrow funnel-shaped structure so as to enable the wings to be inserted into the patient's ear. FIG. 6A is a perspective view of speculum 100 when the speculum's aperture is in a fully closed position. In one example, the diameter of the opening formed by the wings' tips when the speculum's aperture is in a fully closed position is 2 mm.

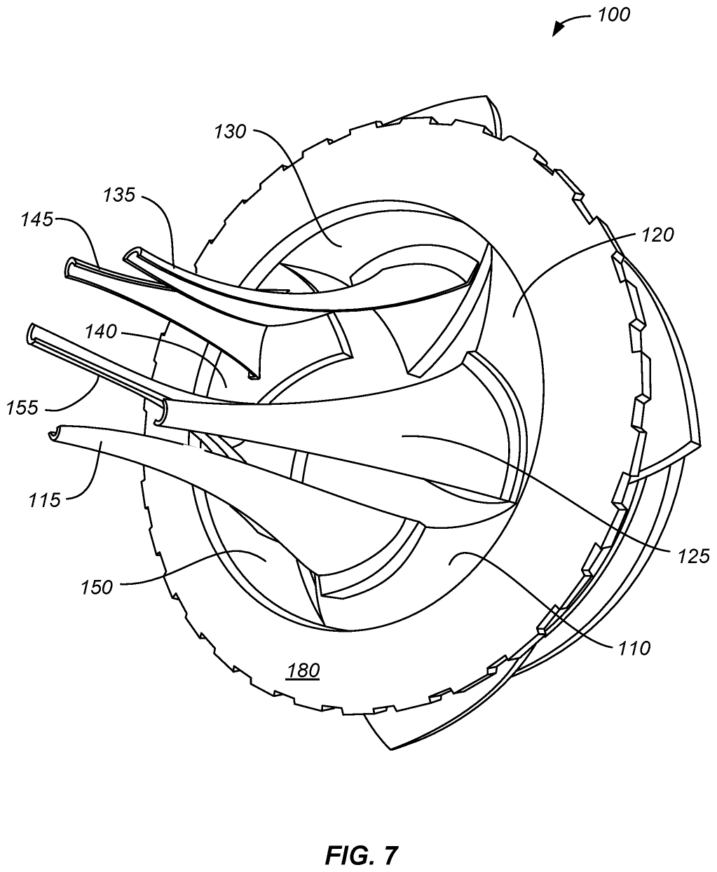

Once inserted into the ear canal, the aperture is opened so as to cause the wings to move away from one another and form a larger opening, as shown in FIG. 7. The doctor/user may continue to open the aperture until the wings' tips come into contact with and gently press against the tissue in the ear canal of the patient being examined or operated on. This advantageously causes the speculum to be held securely in place without using an articulating arm.

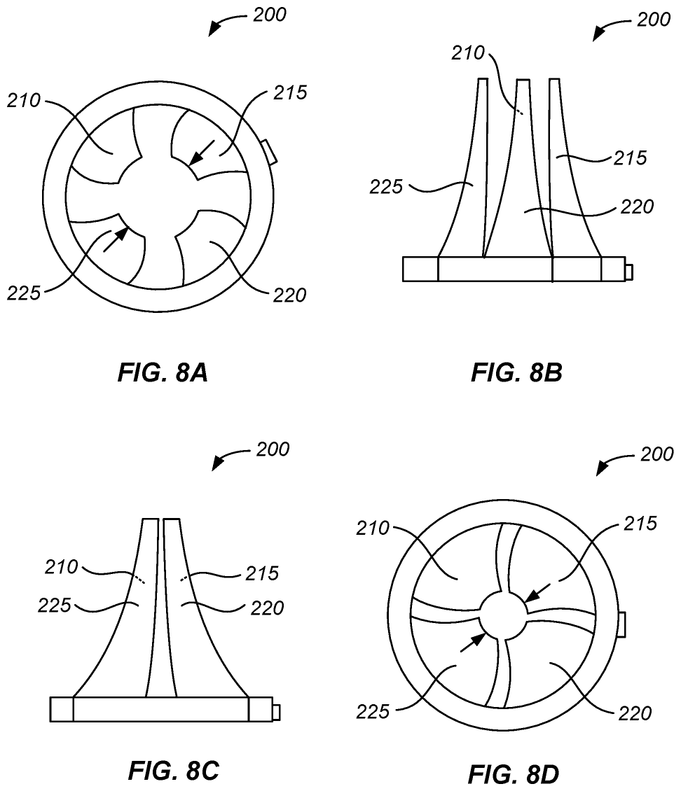

FIGS. 8A and 8B are top and side views, respectively, of a speculum 200 in a fully open position, in accordance with another embodiment of the present disclosure. Embodiment 200 is shown as including, in part, 4 wings 210, 215, 220 and 225, and four associated blades (not shown). In one example, the diameter of the opening formed by the wings' tips when the speculum's aperture is in a fully open position is 6 mm. FIGS. 8C and 8D are top and side views, respectively, of speculum 200, when speculum 200 is placed in a closed position. In one example, the diameter of the opening formed by the wings' tips when the speculum's aperture is in a fully closed position is 2 mm.

By using a speculum, in accordance with embodiments of the present disclosure, a doctor may use both hands to examine or perform an operation on a patient's ear without the use of an articulating arm. Because the diameter of the opening of the speculum's aperture may be adjusted by engaging the speculum's actuating arm, as described above, the speculum may be used to fit any patient, notwithstanding the patient's ear canal size, anatomy or other conditions. An ear speculum, in accordance with embodiments of the present disclosure, therefore, among many other advantages, eliminates the need for (i) a speculum holder during an ear surgery, and/or (ii) a multitude of different ear specula to fit different patients.

The above embodiments of the present invention are illustrative and not limitative. Embodiments of the present invention are not limited by the number of blades or wings of the aperture, the length/shape/positioning of the wings, and the like. Other additions, subtractions or modifications are obvious in view of the present disclosure.

1. A speculum comprising:
an iris aperture comprising an actuator and a plurality of blades adapted to vary an opening of the aperture in response to a force applied to the actuator, each blade comprising a wing extending outwardly from the aperture, each wing having a base that is larger than the wing's tip, each wing having a curved surface along at least a portion of the wing's height.

2. The speculum of claim 1 wherein each blade and its associated wing are formed from a same material shaped to include the blade and its associated wing in a single piece of the material.

3. The speculum of claim 1 wherein the plurality of blades are made from a first material that is different from a second material from which the plurality of wings are made.

4. The speculum of claim 1 wherein after being inserted into a patient's ear canal, a force applied to the actuator causes the plurality of wings to rotate and increase an opening between the wing's tips, wherein the force further causes the plurality of wings to make contacts with tissue in the patient's ear canal so as to securely hold the speculum in place.

5. The speculum of claim 1 wherein said portion of a wing's height is an entirety of the wing's height.

6. The speculum of claim 1 wherein said curved surface is defined by a paraboloid.

7. The speculum of claim 1 wherein said curved surface is defined by an ellipsoid.

8. The speculum of claim 1 wherein said material is selected from a group consisting of plastic and stainless steel.

9. The speculum of claim 1 wherein the speculum comprises at least 4 blades.

10. The speculum of claim 1 wherein the iris aperture comprises a ratchet adapted to adjust and maintain the aperture's opening so as to lock the plurality of wings when inserted into a patient's ear.

11. The speculum of claim 1 wherein the iris aperture is a friction-based aperture adapted to adjust and maintain the aperture's opening via friction so as to lock the plurality of wings when inserted into a patient's ear.

12. A method of forming a speculum, the method comprising:
forming an iris aperture comprising an actuator and a plurality of blades adapted to vary an opening of the aperture in response to a force applied to the actuator, each blade comprising a wing extending outwardly from the aperture, each wing having a base that is larger than the wing's tip, each wing having a curved surface along at least a portion of the wing's height.

13. The method of claim 14 wherein each blade and its associated wing are formed from a same material shaped to include the blade and its associated wing in a single piece of the material.

14. The method of claim 14 wherein the plurality of blades are made from a first material that is different from a second material from which the plurality of wings are made.

15. The method of claim 14 wherein after being inserted into a patient's ear canal, a force applied to the actuator causes the plurality of wings to rotate and increase an opening between the wing's tips, wherein the force further causes the plurality of wings to make contacts with tissue in the patient's ear canal so as to securely hold the speculum in place.

16. The method of claim 14 wherein said portion of a wing's height is an entirety of the wing's height.

17. The method of claim 14 wherein said curved surface is defined by a paraboloid.

18. The method of claim 14 wherein said curved surface is defined by an ellipsoid.

19. The method of claim 14 wherein said material is selected from a group consisting of plastic and stainless steel.

20. The method of claim 14 wherein the speculum comprises at least 4 blades.

21. The method of claim 1 wherein the iris aperture comprises a ratchet adapted to adjust and maintain the aperture's opening so as to lock the plurality of wings when inserted into a patient's ear

22. The method of claim 14 wherein the iris aperture is a friction-based aperture adapted to adjust and maintain the aperture's opening so as to lock the plurality of wings when inserted into a patient's ear.
